(19) United States
(12) Patent Application Publication
(10) Pub. No.: US 2021/0268042 A1
(43) Pub. Date: Sep. 2, 2021

(54) ANTI-INFLUENZA VIRUS AGENT FOR SUPPRESSING AGGRAVATION OF INFLUENZA

(71) Applicants: THE KITASATO INSTITUTE, Tokyo (JP); THE FOOD SCIENCE INSTITUTE FOUNDATION, Tokyo (JP); MEIJI CO., LTD., Tokyo (JP)

(72) Inventors: Takayuki NAGAI, Tokyo (JP); Hiroaki KIYOHARA, Tokyo (JP); Shinji JINNO, Tokyo (JP); Daisuke KOZUTSUMI, Tokyo (JP)

(73) Assignees: THE KITASATO INSTITUTE, Tokyo (JP); THE FOOD SCIENCE INSTITUTE FOUNDATION, Tokyo (JP); MEIJI CO., LTD., Tokyo (JP)

(21) Appl. No.: 17/255,056

(22) PCT Filed: Jul. 3, 2019
(86) PCT No.: PCT/JP2019/026399

§ 371 Date: Dec. 22, 2020

(30) Foreign Application Priority Data
Jul. 3, 2018 (JP) 2018-127101

(51) Int. Cl. A61K 35/745; A61P 31/16

(52) U.S. Cl. CPC A61K 35/745; A61P 31/16

(57) ABSTRACT

The present invention relates to an anti-influenza virus agent comprising Bifidobacterium bifidum as an active ingredient.

### TECHNICAL FIELD

The present invention relates to anti-influenza virus agents for suppressing aggravation of influenza.

### BACKGROUND ART

Influenza is an infectious disease caused by infection with influenza virus. Influenza virus has extremely high infectivity, and spreads infection among approximately 10 million people every year in Japan. Influenza is also epidemic, and thus is transmitted to a number of people in a short period of time upon an onset of epidemic. Elderly people or chronic disease patients affected with influenza have a high probability of the aggravation of influenza, and are at higher risk of complications such as pneumonia, which can even lead to death.

Anti-influenza drugs such as oseltamivir (the brand name: Tamiflu (registered trade mark)) are commonly used for the treatment of influenza. While oseltamivir needs an early administration within 48 hours of onset, too early timing of testing may result in no detection of the virus and fail to make a diagnosis of influenza, making it difficult to administer oseltamivir at an appropriate timing. Therefore, there is a demand for developing a product which can be easily ingested like food products, and can be used for suppressing tire aggravation of influenza upon infection with influenza virus by preliminary ingestion.

Patent Literature 1 discloses that Bifidobacterium lactis strain BL-04 is effective for treating or preventing respiratory diseases such as rhinitis and bronchitis.

Patent Literature 2 discloses a therapeutic or prophylactic composition containing Bifidobacterium longum NCC2705 for infections and immune system-related disorders including infections.

Bifidobacterium bifidum strain OLB6378, a bifidobacterium, has been confirmed to have a stimulatory effect on intestinal mucosal immunity, and a prophylactic or therapeutic effect on allergy (Non Patent Literature 1 and Patent Literature 3).

However, it has not been revealed whether these microorganisms suppress the aggravation of influenza.

### CITATION LIST

### Patent Literature

Patent Literature 1 JP Patent Publication No. 2014-517003 A
Patent Literature 2 JP Patent Publication No 2012-526752 A
Patent Literature 3 JP Patent Publication No 2006-273852 A

### Non Patent Literature

Non Patent Literature 1 Tanaka K. et al., Nutrients. (2017) 9, 195; doi:10.3390/nu9030195

### SUMMARY OF INVENTION

### Technical Problem

A problem of the present invention is to provide an anti-influenza virus agent for suppressing aggravation of influenza.

### Solution to Problem

The present inventors intensively studied to address the problem described above, and as a result, have found out a Bifidobacterium bifidum strain that is capable of suppressing aggravation of influenza, and achieved to complete the present invention.

Specifically, the present invention encompasses the followings.

[1] An anti-influenza virus agent comprising Bifidobacterium bifidum as an active ingredient.
[2] The anti-influenza virus agent according to the above [1], wherein the Bifidobacterium bifidum has a suppressing effect on growth of influenza virus in respiratory tract.
[3] The anti-influenza virus agent according to the above [1] or [2], wherein the Bifidobacterium bifidum is Bifidobacterium bifidum strain OLB6378 (Accession No. NITE BP-31).
[4] The anti-influenza virus agent according to any of the above [1]-[3], which is for use in suppressing aggravation of influenza.
[5] The anti-influenza virus agent according to any of the above [1]-[4], wherein the Bifidobacterium bifidum is selected from the group consisting of a culture, a heat-treated product, a dried product, and a concentrate of Bifidobacterium bifidum bacterial cells, and a combination of two or more thereof.
[6] A food product comprising the anti-influenza virus agent according to any of the above [1]-[5], wherein said food product is for use in suppressing aggravation of influenza.
[7] A medicament comprising the anti-influenza virus agent according to any of the above [1]-[5], wherein said medicament is for use in suppressing aggravation of influenza.

### Advantageous Effect of Invention

The present invention enables suppression of the aggravation of influenza upon infection with influenza virus.

The present specification includes the contents of Japanese Patent Application No. 2018-127101, from which the present application claims priority.

### BRIEF DESCRIPTION OF DRAWINGS

FIG. 1 indicates an effect of strain OLB6378 on survival rates of mice having lower respiratory tract infection with influenza virus. *p=0.05 (relative to the water-administered group), n=9. In the Figure, the survival rates are shown as a square for the oseltamivir-adminstered group (positive control group), a closed circle for the OLB6378-administered group, and an open circle for the water-administered group.

FIG. 2 FIG. 2 indicates an effect of strain OLB6378 on infectious virus titers in bronchoalveolar lavage fluid of mice having lower respiratory tract infection with influenza virus. **p<0.01, ***p<0.001 (relative to the water-administered group).

### DESCRIPTION OF EMBODIMENTS

The present invention will be now described in detail below.

The present invention relates to use of Bifidobacterium bifidum, preferably Bifidobacterium bifidum having a suppressing effect on growth of influenza virus m respiratory tract, as an anti-influenza virus agent.

“Bifidobacterium bifidum” as used in the present invention is one species of genus Bifidobacterium. In the present invention, classification of bacteria into Bifidobacterium bifidum can be made according to a common taxonomic criteria. A Bifidobacterium bifidum strain to be used in the present invention is not particularly limited, but preferably has a suppressing effect on growth of influenza virus in respirators' tract.

In the present invention, Bifidobacterium bifidum “having a suppressing effect on growth of influenza virus in respiratory tract” means that the Bifidobacterium bifidum has an ability to suppress the growth of influenza virus in respiratory tract, i.e., upper respiratory tract and/or tower respiratory tract. “Upper respiratory tract” typically refers to nasal cavity, pharynx, and larynx. “Lower respiratory tract” typically refers to trachea, bronchi, and lungs. Bifidobacterium bifidum to be used in the present invention can suppress the growth of influenza vims in respiratory tract, e.g., in at least one (e.g., two or more) of nasal cavity, pharynx, larynx, trachea, bronchi, and lungs.

In the present invention, “suppression” of the growth of influenza virus in respiratory tract denotes that administration of a test substance (in this context. Bifidobacterium bifidum) results in a decrease of infectious virus titer of influenza vims collected front respiratory tract compared to that in administration with water instead of the test substance. The decrease of infectious virus titer is preferably a statistically significant decrease, for example, it may be a decrease with a statistically significant difference shown by Dannett's test. The decrease of infectious virus titer due to administration of a test substance may also be a decrease by 30% or more, or 40% or more, e.g., 50% or more in average, compared to that due to administration with water instead of the test substance.

Examples of Bifidobacterium bifidum which can be used in the present invention include Bifidobacterium bifidum strain OLB6378. Bifidobacterium bifidum strain OLB6378 was deposited under Accession No. NITE P-31 with National Institute of Technology and Evaluation, Patent Microorganisms Depositary (NPMD) (#122, 2-5-8 Kazusakamatari, Kisarazu-shi, Chiba, Japan, postal code: 292-0818) on Oct. 26, 2004 (the original deposition date), and then transferred from the original deposit to the deposit under the Budapest Treaty on Jan. 18, 2006, under Accession No. NITE BP-31 into which the original accession number was changed. Bifidobacterium bifidum strain OLB6378 has a suppressing effect on growth of influenza vims in respirators' tract. Note that the current depositor of Bifidobacterium bifidum strain OLB6378 is Meiji Co., Ltd.

The present invention relates to an anti-influenza virus agent comprising Bifidobacterium bifidum, preferably Bifidobacterium bifidum having a suppressing effect cm growth of influenza virus in respiratory tract, as an active ingredient. The anti-influenza virus agent according to the present invention may contain an effective amount of such Bifidobacterium bifidum.

The anti-influenza virus agent according to the present invention can be used tor suppressing the growth of influenza virus, typically suppressing the growth of influenza virus in respiratory tract. An influenza virus to be targeted for application of the agent may be, but are not limited to, type A (such as Russian flu, pdm09 flu, or Hong Kong flu), type B, type C, or a new-type. Type A influenza virus may fee, without limitation, a subtype such as H1N1, H1N2, H2N2, H2N3, H3N2, H5N1, H5N6, H7N7, H7N9, or H10N8.

In the present invention. Bifidobacterium bifidum can be used in any form in the anti-influenza virus agent, as well as food product and medicament as described below, as long as it retains an anti-influenza virus effect, preferably a suppressing effect on growth of influenza virus in respiratory tract and/or an effect of suppressing aggravation of influenza as described below. In one embodiment, Bifidobacterium bifidum to be used in the anti-influenza virus agent, food product, and medicament may be bacterial cells prepared by any method, and/or may be any processed product of bacterial cells, for example, selected from the group consisting of a culture, a heat-treated product, a dried product (e.g., a lyophilized product such as lyophilized powder), and a concentrate of bacterial cells of Bifidobacterium bifidum, and any combination of two or more thereof. Bifidobacterium bifidum may be dead bacterial cells or living bacterial cells. In one embodiment. Bifidobacterium bifidum may be a heat-treated bacterial liquid concentrate or a lyophilized product thereof. In one embodiment, Bifidobacterium bifidum can be subjected to heat treatment at a temperature of 70 to 90° C. (e.g., 75 to 85° C.), and for example, it may be heated for 5-30 minutes, but without limitation to these conditions.

The above-mentioned Bifidobacterium bifidum according to the present invention and anti-influenza virus agent comprising it have an effect of suppressing aggravation of influenza. In the present invention, the “aggravation of influenza” refers to becoming severe medical conditions upon infection with influenza virus, which are likely to cause a decrease of survival rate. Examples of the severe medical conditions include complications such as bronchitis, pneumonia, influenza-associated encephalopathy, encephalitis, otitis media, and febrile seizure: and high fever, severe general malaise and the like. In the present invention, the expression “suppressing aggravation of influenza” encompasses reducing a probability of developing such severe medical conditions, preventing development of the severe medical conditions or alleviating the severity, and facilitating early recovers' from the severe medical conditions, upon infection with influenza virus. Thus, the anti-influenza virus agent according to the present invention may be for use in suppressing aggravation of influenza. Suppression of aggravation of influenza may be analyzed, for example, based on an improvement of survival rate after infection with influenza virus, as an indicator. If administration of a test substance (in this context. Bifidobacterium bifidum) results in an increase of survival rate after infection with influenza virus compared to that in administration of water instead of the test substance, it indicates that the test substance suppressed aggravation of influenza. The increase of survival rate is preferably a statistically significant increase, for example, it may be an increase with a statistically significant difference shown by Log-rank test with Kaplan-Meier method. The anti-influenza virus agent (and the above-mentioned Bifidobacterium bifidum) according to the present invention suppresses aggravation of influenza and thereby can reduce the risk of aggravation of influenza.

The present invention also provides a food product or a medicament, which comprises the anti-influenza virus agent (or the above-mentioned Bifidobacterium bifidum) according to the present invention. The food product and medicament comprising the anti-influenza virus agent (or the above-mentioned Bifidobacterium bifidum) according to the present invention can suppress aggravation of influenza in a subject that ingests or is administered with it. The food product and medicament according to the present invention can suppress the growth of influenza virus in respiratory tract. The food product and medicament according to the present invention may be for use in suppressing aggravation of influenza. The food product and medicament according to the present invention may be for use in reducing the risk of aggravation of influenza.

The anti-influenza agent, food product, and medicament according to the present invention may contain an orally acceptable additive for ingestion or pharmaceutically acceptable additive. Examples of orally acceptable additive for ingestion or pharmaceutically acceptable additives include, but not limited to, carriers (such as solid or liquid carriers), excipients, surfactants, binders, disintegrants, lubricants, solubilizing agents, suspending agents, coating agents, colorants, flavoring agents, preservatives, buffer agents, pH adjusters, diluents, stabilizing agents, propellants, antioxidants, thickeners, and sweeteners. These additives can be used alone or in a combination of two or more, and can be appropriately used depending on the dosage form of a preparation, and/or the kind or form of a food product. Examples of carriers include water, orally acceptable organic solvent, collagen, polyvinyl alcohol, polyvinylpyrrolidone, carboxyvinyl polymer, sodium alginate, water-soluble dextran, water-soluble dextrin, sodium carboxymethyl starch, pectin, xanthan gum, gum arabic, casein, gelatin, agar, glycerin, propylene glycol, polyethylene glycol vaseline, paraffin, stearyl alcohol stearic acid, human serum albumin, mannitol, sorbitol lactose, and orally acceptable surfactants.

The “food product” in the context of the present invention encompasses, but not particularly limited to, beverage products, toed products other than beverage products, and functional food products. The “food product” in the context of the present invention may be a food composition. The kind of the food product according to the present invention is not particularly limited, and examples of beverages include fermented milk (such as yogurt drinks), lactic acid bacteria drinks, milk beverages (such as coffee-flavored milk and fruit-flavored milk), tea-based beverages (such as green tea, black tea, and oolong tea), fruit/vegetable-based beverages (beverages containing fruit juice of orange, apple, grape or the like and/or vegetable juice of tomato, carrot or the like), alcoholic beverages (such as beer, low-malt beer, and wine), oral rehydration solutions, carbonated beverages, soft drinks, and water. For manufacturing methods and the like of various beverages, conventional guidebooks such as “Latest Soft Drinks” (2003) (Korin, Co., Ltd.), for example, can be referred to. Examples of food product include fermented milk (such as set-type yogurt, soft yogurt, and cheese), dairy products, powdered formula (powdered formula milk), confectioneries (such as chocolate, gummi candy, chewing gum, biscuits, cookies, jelly, tablets, and cold/frozen desserts), and instant foods. For manufacturing methods and the like of various food products, conventional guidebooks can be referred to.

The food product according to the present invention may be a functional food product. The “functional food product” of the present invention means a food product having a given functionality for a living body, and encompasses overall so-called health foods, for example, foods with health claims, such as foods for specified health uses (including conditional foods for specified health use) and foods with nutrient function claims, foods with function claims, and foods for special dietary uses, as defined in Japan; as well as nutritional supplement foods, health supplement foods, supplement preparations (in various dosage forms such as tablets, coated tablets, sugar-coated tablets, capsules, and liquids) and foods for beauty (such as weight-reducing foods), five functional food product of the present invention also encompasses health foods to which a health claim based on the food standards by Codex (Joint FAO/WHO Codex Alimentarius Commission) is applied.

The functional food product of live present invention may be a solid preparation such as tablets, granule, powder, pills, or capsules; a liquid preparation such as liquid, suspension, or syrup; or gel paste or the like, or may be in a common form of food or drink (such as a beverage, yogurt, or confectionery).

The food product according to the present invention may be intended for a subject susceptible to influenza or susceptible to aggravation of influenza when affected with influenza. The food product according to the present invention may be intended for ingestion by newborns (including particularly neonates, healthy newborns, premature newborns, preterm newborns, low birth weight newborns and the like), infants, toddlers, children, sick people, otherwise healthy people with weak constitutions, otherwise healthy people in poor physical condition, expectant and nursing mothers, elder people (typically aged 60 years or more), chronic disease patients (e.g., patients with cardiac disease, respiratory disease, renal dysfunction, immune dysfunction, or metabolic disease such as diabetes) and the like: as well as household and other close contacts thereof (such as healthcare professionals, childcare and educational professionals, and livelihood supportive professionals). The food product according to the present invention may be food for special dietary use, such as food for sick people, powdered milk for expectant and nursing mothers and lactating mothers, powdered formula for infants, infant formula, food for elder people, or food tor nursing-care. The food product according to the present invention may be powdered milk, fluid diet, semifluid diet, powdered fluid diet, nursing-care food, toddler food, thickened food, energy paste, powdered nutrient, nutrient-enriched food, liquid formula, or the like.

The Bifidobacterium bifidum or anti-influenza virus agent of the present invention can be contained in the food product by any appropriate method available to a skilled person in the art. For example, the Bifidobacterium bifidum of the present invention may be prepared in a form of liquid, gel, solid, powder, or granule, and then used to prepare a food product. Alternatively, the Bifidobacterium bifidum or anti-influenza virus agent of the present invention may be directly mixed with or dissolved in ingredients of food or drink. The Bifidobacterium bifidum or anti-influenza virus agent of the present invention may be applied to, coated on, permeated into, or sprayed on food or drink. The Bifidobacterium bifidum or anti-influenza virus agent of the present invention may be evenly dispersed or unevenly distributed into food or drink. The Bifidobacterium bifidum or anti-influenza virus agent of the present invention may be encapsulated to prepare capsules or the like. The Bifidobacterium bifidum or anti-influenza virus agent of the present invention may be wrapped with edible film, food coaling agent or the like. The Bifidobacterium bifidum or anti-influenza virus agent of the present invention may also be added with an appropriate excipient and the like, and then shaped into tablets or the like. Food or drink containing the Bifidobacterium bifidum or anti-influenza virus agent of the present invention may be further processed, and such processed product also falls within the scope of the present invention. In one embodiment, a heat-treated bacterial cell concentrate of the Bifidobacterium bifidum of the present invention may be added to food ingredients (such as rehydrated powdered formula, or fluid diet ingredients), heat sterilized, and concentrated and dried.

The amount of the Bifidobacterium bifidum or anti-influenza virus agent of the present invention to be used in the food product is not particularly limited, and may vary depending on the circumstances. The specific amount of the Bifidobacterium bifidum or anti-influenza vims agent of the present invention can be appropriately determined by a skilled person in the art in consideration of the kind or required taste or texture of the food product. Commonly, the amount of the Bifidobacterium bifidum can be 0.0001 to 100% by weight (i.e., by mass), e.g., 0.0005 to 100% by weight (i.e., by mass) or 0.001 to 99% by weight (i.e., by mass), as die ratio of dry weight equivalent of bacterial cells of Bifidobacterium bifidum to be used (solid content composed of bacterial cells) relative to the total weight of the food product. In one embodiment, 0.0001 to 100% by weight (i.e., by mass), e.g., 0.001 to 99% by weight (i.e., by mass), 0.01 to 10% by weight (i.e., by mass), or 0.01 to 0.1% by weight (i.e., by mass) of Bifidobacterium bifidum, as the ratio of dry weight equivalent of bacterial cells of Bifidobacterium bifidum to be used (solid content composed of bacterial cells) relative to the total weight of die food product may be contained in a solid food product, such as powdered food (such as powdered formula or powdered seasoning), granular food, flaked food, readily disintegrating solid food or the like. Such solid food product may be intended to be dissolved in a drinkable liquid medium such as water or drink to prepare a beverage (such as liquid formula or oral rehydration solution), liquid food (such as soup or curry), liquid seasoning, fluid diet, semifluid diet, thickened food, or the like, and then be ingested. In another embodiment, 0.0001 to 100% by weight (i.e., by mass), e.g., 0.0005 to 10% by weight (i.e., by mass) or 0.001 to 5% by weight (i.e., by mass) of Bifidobacterium bifidum, as the ratio of dry weight equivalent of bacterial cells of Bifidobacterium bifidum to be used (solid content composed of bacterial cells) relative to the total w eight of the food product may be contained in a liquid or semiliquid food product, e.g., snip, curry, fermented milk, beverage (such as liquid formula or oral rehydration solution), liquid seasoning, fluid diet, semifluid diet, thickened food or the like. The foregoing amount of Bifidobacterium bifidum being expressed based on dry weight equivalent of bacterial cells docs not exclusively mean that Bifidobacterium bifidum is contained in the food product or the like in a form of dried bacterial cells. The Bifidobacterium bifidum of the present invention can be used in the food product or the like in any form as long as it retains the anti-influenza virus effect, in particular, a suppressing effect on growth of influenza virus in respiratory tract and/or an effect of suppressing aggravation of influenza.

The food product according to the present invention may further contain any food ingredient(s). The food product according to the present invention may contain water, protein, carbohydrate, lipid, vitamins, minerals, amino acids, organic acids, organic bases, fruit juice, flavors, or the like. Examples of proteins include animal and plant proteins such as whole milk powder, skimmed milk powder, partially skimmed milk powder, casein, whey powder, whey protein, whey protein concentrate, whey protein Isolate, α-casein, β-casein, κ-casein, β-lactoglobulin, α-lactoalbumin, lactoferrin, soybean protein, hen egg protein, and meat proteins; hydrolysates thereof; and various milk-derived ingredients such as butter, whey mineral, cream, whey, non-protein nitrogen, sialic acids, phospholipids, and lactose. Examples of carbohydrates include common saccharides, modified starches (such as dextrin, soluble starch, British starch, oxidized starch, starch ester, and starch ether), and dietary fiber. Examples of lipids include animal fats and oils such as lard, fish oil and the tike, and fractionated oil, hydrogenated oil, interesterified fed oil and the like thereof; and vegetable fats and oils such as palm oil, sunflower oil, corn oil, canola oil, and coconut oil, and fractionated oil, hydrogenated oil, interesterified oil and the like thereof. Examples of vitamins include vitamin A, carotenes, vitamin B group, vitamin C, vitamin D group, vitamin E, vitamin K group, vitamin P, vitamin Q, niacin, nicotinic acid, pantothenic acid, biotin, inositol, choline, and folic acid, and examples of minerals include calcium, potassium, magnesium, sodium, cupper, iron, manganese, zinc, selenium, and whey mineral. Examples of organic acids include malic acid, citric acid, lactic acid, and tartaric acid. These ingredients can be used alone or in a combination of two or more.

The medicament according to the present invention may be any dosage form, including solid preparations such as tablets, granules, powders, pills, or capsules: gels; and liquid preparations such as liquids, suspensions, or syrups. The “medicament” in the context of the present invention may be a pharmaceutical composition.

The anti-influenza agent, food product, and medicament according to the present invention may further contain any other substance having anti-influenza effect.

The anti-influenza agent, food product, and medicament according to the present invention may be intended for oral administration or parenteral administration, but preferably is for oral administration.

The present invention also provides a method of treating or preventing influenza, the method comprising administering the anti-influenza agent, food product, or medicament according to the present invention to a subject. The present invention also provides a method of suppressing aggravation of influenza, the method comprising administrating the anti-influenza agent, food product, or medicament according to the present invention to a subject. The term “administration” in the context of the present invention can encompass all of “ingestion” for food product, “administration” for medicament, and “treatment” of cells or tissues in vivo. In the present invention, “oral administration” (also referred to as “oral or enteral administration”) shall also include administration or ingestion from mouth, as well as administration by tube feeding via a nasal tube, a gastrostomy or enterostomy tube or the like. The present invention also provides a method of reducing the risk of aggravation of influenza, the method comprising administering the anti-influenza agent, food product, or medicament according to the present invention to a subject. The present invention further provides use of the above-mentioned Bifidobacterium bifidum according to the present invention, e.g., Bifidobacterium bifidum strain OLB6378, in the manufacture of the anti-influenza agent, food product, or medicament according to the present invention.

The dosage of the anti-influenza agent, food product, anti medicament according to the present invention can be modified over a wide range at discretion of a skilled person in the art in consideration of age and body weight of a subject to be administered therewith, route of administration, number of doses, and the like. The dosage of the Bifidobacterium bifidum according to the present invention may be, but not particularly limited to, for example, an amount equivalent to 1×10⁵ to 1×10¹¹ cells, or 1×10⁶ to 1×10¹⁰ cells, or 1×10⁷ to 1×10¹⁰ cells per dose.

In one embodiment, the anti-influenza agent, food product, or medicament according to the present invention may be administered to a subject once or more a day, e.g., twice or more a day. The anti-influenza agent, food product, or medicament according to the present invention may be continuously administered to a subject, for example, it may be administered daily. The anti-influenza agent, food product, or medicament according to the present invention may be administered to a subject for one week, two weeks, three weeks, or four weeks or more (such as a year or more).

The anti-influenza agent, food product, anti medicament according to the present invention can be administered to a subject regardless before or after infection with influenza virus, but it is preferably to be administered before infection with influenza virus.

The subjects to be administered with the anti-influenza agent, food product, or medicament according to the present invention may be any animals, preferably mammals including primates such as human, gorillas, or chimpanzees; dogs, cats, mice, rats, rabbits, horses, bovines, sheep, and goats; or cells thereof. The subjects to be administered with the anti-influenza agent, food product, or medicament according to the present invention may be a subject susceptible to influenza or susceptible to aggravation of influenza when affected with influenza. In a preferred embodiment, human subjects may be newborns, infants, toddlers, children, sick people, otherwise healthy people with weak constitutions, otherwise healthy people in poor physical condition, expectant and nursing mothers, elder people, chronic disease patients and the like: as well as families and other close contacts thereof as described above, but the subjects are not limited thereto. The anti-influenza agent, food product, and medicament according to the present invention can be used for a wide range of subjects at a risk of suffering from influenza.

### EXAMPLES

The present invention will be now described more specifically with Examples below. However, the technical scope of the present invention is not limited to these Examples.

### Example 1 Preparation of Powdered OLB6378 Concentrate

Bifidobacterium bifidum strain OLB6378 (Accession No. NITE BP-31) was anaerobically cultured in a whey protein hydrolysate medium (a medium containing enzymatically-degraded whey protein as the main nitrogen source), with neutralizing tire medium, thereby preparing a bacterial liquid concentrate. The bacterial liquid concentrate was incubated at 80° C. for no less than 10 minutes, and then lyophilized. The resulting lyophilized product showed a bacterial concentration of more than 2.5×10¹¹ cells/g. The lyophilized product of the heat-treated strain OLB6378 thus prepared was referred to as “powdered OLB6378 concentrate,” and tested for anti-influenza virus effect as described below.

### Example 2 Evaluation of Anti-Influenza Virus Effect of Powdered OLB6378 Concentrate

Anti-influenza virus effect of the powdered OLB6378 concentrate prepared in Example 1 was evaluated in term of “survival rate” and “infectious virus titer (viral growth level) in respiratory tract” after infection with influenza virus.

### a) Survival Rate

BALB/c mice (female, 8-week-old) (CLEA Japan, Inc.) were orally administered daily with water (water-administered group) or 20% suspension of powdered OLB6378 concentrate (OLB6378/bifidobacterium-administered group) at a dose of 0.2 ml/animal/day for 4 weeks. Three weeks after the start of the oral administration, the mice were anesthetized by intraperitoneal administration of anesthetic Somnopentyl® (5 mg/kg body weight of the active ingredient, sodium pentobarbital), and then nasally inoculated with 20 μl of suspension of mouse-adapted influenza virus strain A/PR/8/34 (MINI) (2×LD₅₀, which corresponds to a double amount of median lethal dose; in 0.1% BSA-PBS) into right nasal cavities to induce lower respiratory tract infection with influenza virus. As a positive control, anti-influenza drug “oseltamivir” (1 mg/kg body weight/day), instead of administration of water or 20% suspension of powdered OLB6378 concentrate, was orally administered from 1 hour before to 6 days after inoculation with influenza virus in a similar manner to that described above. Survival rate of the mice was recorded for 21 days after the start of the virus inoculation. Survival analysis was performed by log-rank test with Kaplan-Meier method.

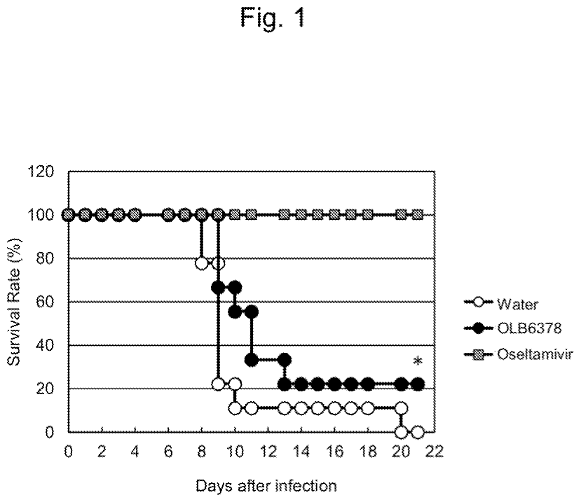

The results are shown in FIG. 1. In the mice orally administered with water (water-administered group), dead mice began to be observed 8 days after virus inoculation (infection), survival rate sharply reduced to about 11% within 10 days after the infection, and all the mice died 20 days after the infection (survival rate of 0%). In contrast, in the mice orally administered with 20% suspension of powdered OLB6378 concentrate (OLB6378-administered group), though dead mice began to be observed 9 days after virus inoculation (infection), survival rate of about 56% was still shown 10 days after the infection, and survival rate of about 22% was shown even 21 days after the infection. The improvement in survival rate by administration with powdered OLB6378 concentrate was statistically significant compared to the water-administered group (p=0.05, FIG. 1).

Meanwhile, no dead mouse was observed in the positive control group in which mice were orally administered with anti-influenza drug “oseltamivir” and infected in lower respirator) tract, with influenza virus (FIG. 1).

### b) Infectious Virus Titer (Viral Growth Level)

BALB/c mice (female, 8-weeks-old) (CLEA Japan. Inc.) were orally administered daily with water (water-administered group) or 20% suspension of powdered OLB6378 concentrate (OLB6378-administered group; test group) at a dose of 0.2 ml/animal/day for 25 days (from 21 days before to 3 days alter virus inoculation). Three weeks alter the start of the oral administration, the mice were anesthetized by intraperitoneal administration of anesthetic Somnopentyl® (5 mg/kg body weight of the active ingredient, sodium pentobarbital), and then nasally inoculated with 20 μl of suspension of mouse-adapted influenza virus strain A/PR/8/34 (H1N1) (2×LD₅₀, which corresponds to a double amount of median lethal dose; in 0.1% BSA-PBS) into right nasal cavities to induce infection with influenza virus. As a positive control, anti-influenza drug “oseltamivir” (1 mg/kg body weight/day), instead of administration of water or 20% suspension of powdered OLB6378 concentrate, was orally administered from 1 hour before to 3 days after inoculation with influenza virus in a similar manner to that described above. Four days after the virus inoculation, bronchoalveolar lavage fluid (BALF) was prepared from the mice. Infectious virus titer in the bronchoalveolar lavage fluid was determined with plaque assay. Specifically, MDCK cells (a cell line from canine renal tubule epithelial cells) were exposed to the bronchoalveolar lavage fluid, anti then the number of plaques that are formed due to cell degeneration by virus infection was counted to evaluate the number of the formed plaque per 1 mL of the bronchoalveolar lavage fluid as infectious virus titer. Statistical analysis was performed using analysis of variance (ANOVA) and Dunnett's test.

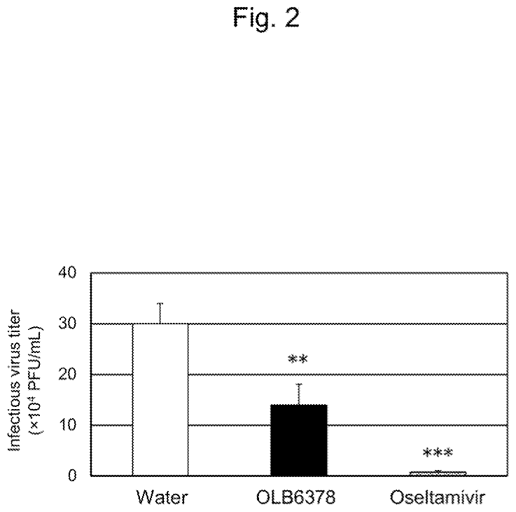

The results are shown in FIG. 2. Four days after influenza virus infection, the mice orally administered with water (water-administered group) exhibited a high infectious vims titer in the bronchoalveolar lavage fluid: indicating establishment of lower respiratory tract infection. On the other hand, the mice orally administered with 20% suspension of powdered OLB6378 concentrate (OLB6378-administered group) exhibited a statistically significant decrease in infectious virus titer in the bronchoalveolar lavage fluid compared to the water-administered group (decrease of more than 53% compared to water-administered group) (p<0.01. FIG. 2).

Additionally, the positive control group in which mice were orally administered with anti-influenza drug “oseltamivir” and infected in lower respiratory tract with influenza virus also exhibited significant decrease in infectious virus liter in the bronchoalveolar lavage fluid compared to the water-administered group (p<0.001. FIG. 2).

These test results of the survival rate and infectious virus titer (viral growth level) showed that strain OLB6378 suppresses virus growth in bronchus and lung after infection with influenza virus, and also improves survival rale. This shows that strain OLB6378 has anti-influenza vims effect, and particularly provides an effect of suppressing the aggravation of influenza.

### INDUSTRIAL APPLICABILITY

The present invention can be used to provide an anti-influenza product having an effect of suppressing aggravation of influenza. The anti-influenza virus agent of the present invention can suppress aggravation of symptoms of influenza virus infection by routine ingestion of the agent in a form of food product or the like. The present invention can provide an anti-influenza product useful for suppressing aggravation of symptoms of influenza particularly in a subject susceptible to aggravation of influenza, such as a newborn (particularly a neonate), an infant/toddler, a child, a sick person, an elder person, or a chronic disease patient.

All the publications, patents, and patent applications cited in the present specification shall be incorporated by reference in their entirety herein.

1-7. (canceled)

8. A method of treating or preventing influenza in a subject comprising administering an anti-influenza virus agent comprising Bifidobacterium bifidum as an active ingredient, a food product comprising Bifidobacterium bifidum as an active ingredient, or a medicament comprising Bifidobacterium bifidum as an active ingredient to the subject.

9. The method according to claim 8, wherein the Bifidobacterium bifidum has a suppressing effect on growth of influenza virus in respiratory tract.

10. The method according to claim 8, wherein the Bifidobacterium bifidum is Bifidobacterium bifidum strain OLB6378 (Accession No. NITE BP-31).

11. The method according to claim 8, wherein the method suppresses aggravation of influenza.

12. The method according to claim 8, wherein the Bifidobacterium bifidum is selected from the group consisting of a culture, a heat-treated product, a dried product, and a concentrate of Bifidobacterium bifidum bacterial cells, and a combination of two or more thereof.

13. A method of suppressing aggravation of influenza in a subject comprising administering an anti-influenza virus agent comprising Bifidobacterium bifidum as an active ingredient, a food product comprising Bifidobacterium bifidum as an active ingredient, or a medicament comprising Bifidobacterium bifidum as an active ingredient to the subject.

14. The method according to claim 13, wherein the Bifidobacterium bifidum has a suppressing effect on growth of influenza virus in respiratory tract.

15. The method according to claim 13, wherein the Bifidobacterium bifidum is Bifidobacterium bifidum strain OLB6378 (Accession No. NITE BP-31).

16. The method according to claim 13, wherein the Bifidobacterium bifidum is selected from the group consisting of a culture, a heat-treated product, a dried product, and a concentrate of Bifidobacterium bifidum bacterial cells, and a combination of two or more thereof.
